(19) United States
(12) Patent Application Publication
(10) Pub. No.: US 2003/0181753 A1
(43) Pub. Date: Sep. 25, 2003

(54) Method for producing amides or esters

(76) Inventors: Harald Groger, Hanau (DE); Jurgen Sans, Trostberg (DE); Anita Barthuber, Schnaitsee (DE); Roswitha Haindl, Kienberg (DE)

Correspondence Address: FULBRIGHT & JAWORSKI, LLP, 666 FIFTH AVE, NEW YORK, NY, 10103-3198, US

(21) Appl. No.: 10/297,825

(22) PCT Filed: Jun. 12, 2001
(86) PCT No.: PCT/EP01/06655

(30) Foreign Application Priority Data
Jun. 14, 2000 (DE) 100 29 139.2

(51) Int. Cl. C07C 231/02; C07C 69/74; C 07D 4 3/14

(52) U.S. Cl. 560/1; 564/138; 544/212

(57) ABSTRACT

The invention relates to a method for producing amides or esters from carboxylic acids and from an amine constituent or alcohol constituent in the presence of a 1,3,5-triazine and optionally in the presence of an organic solvent and of a tertiary amine. According to the invention, a (bi)cyclic diamine or an adduct formed therefrom with the triazine constituent is used as a tertiary amine in a preferred stoichiometric ratio of diamine to the triazine constituent ranging from 0.30 to 1.10; the stoichiometric ratio of carboxylic acid to the amine constituent or alcohol constituent should range from 0.2 to 5.0, and; the molar ratio of carboxylic acid to the triazine constituent ranges from 0.5 to 1.5. Amino acids such as N-protected amino acids and peptides serve as carboxylic acid constituents and (C-protected) amino acids or a C-protected peptide serve as the amine constituent. 2-chlorine-4,6-dimethoxy-1,3,5-triazine (CDMT) is used as the preferred 1,3,5-triazine, and the N,N′-dimethyl-1,4-piperazine is used as the cyclic diamine. In addition to this method, which can be carried out at temperatures ranging from −80 to +150 ° C. and in the presence of an organic solvent, the invention also relates to adducts comprised of (bi)cyclic diamine and 1,3,5-triazine. Compared to the prior art, higher yields with shorter reaction times are achieved using the described method, and distinctly smaller waste quantities of tertiary amine bases accrue.

The present invention provides a process for preparing amides or esters.

A current, established process for preparing amides or esters which has been described in detail in the literature is the coupling of a carboxylic acid with an amine or alcohol using at least one equivalent of a 1,3,5-triazine as coupling reagent to give the desired amide or ester [Z. J. Kaminski, Tetrahedron Lett. 1985, 26, 2901-2904; Z. J. Kaminski, Synthesis 1987, 917-920; L. Alig et al., EP 0381033, 1990; P. A. Hipskind et al., J. Org. Chem. 1995, 60, 7033-7036; E. C. Taylor et al., J. Org. Chem. 1996, 61, 1261-1266]. 2-Chloro-4,6-dimethoxy-1,3,5-triazine (CDMT) has proven to be the most efficient triazine component. An additional requirement in this process is the presence of stoichiometric amounts (at least one equivalent) of a base in the form of a tertiary amine, and N-methylmorpholine is used almost exclusively.

In this context, the term “equivalent” refers by definition to the molar amounts of the quantity being considered (for example of the 1,3,5-triazine or tertiary amine) based on the molar proportion of the component used which is relevant to the calculation of the theoretical yield of the amide product, or, when the component relevant to the calculation of the theoretical yield of the amide product contains more than one reactive functional group (for example in the case of a dicarboxylic acid), of the reactive functional group.

The abovementioned processes lead to the desired products in good to very good yields and have already been described for a large number of highly varying applications. For instance, a multitude of pharmaceutically interesting amides, in particular peptides, and esters are accessible by this route. The carboxylic acid used for peptide synthesis may be an N-protected amino acid or C-terminal peptide, and the amine used is typically a carboxyl-protected amino acid or an N-terminal peptide.

The coupling of such carboxylic acids or amines leads to the industrially particularly interesting compound class of the peptides, which is why this coupling method is widely used and is of high commercial interest. Alternatively, the 1,3,5-triazine and a tertiary amine may also be replaced by a corresponding adduct of these two components [M. Kunishima et al., Tetrahedron 1999, 55, 13159-13170], although this requires an additional isolation step.

However, despite the varying applications in the field of amide and peptide or ester synthesis, the above-described process has some serious disadvantages:

For instance, the base N-methylmorpholine which is customarily used has a relatively high molecular mass and correspondingly leads to large amounts of waste. The use of a tertiary amine having a smaller molar mass would therefore be desirable for reasons of atom economy and also from an ecological point of view, not least on account of the resulting substantially reduced amounts of waste in industrial applications. Unfortunately, all experiments using bases of small molar mass have hitherto been unsuccessful.

A further disadvantage of the existing processes is in the workup stages: although the hydrochloride formed from the tertiary amine is predominantly soluble in water, it also has a marked solubility in organic solvents. Although this could be reduced by introducing a second ionic charge into the tertiary amine molecule, for example to form a dihydrochloride, this would require the presence of a second base function which could itself be protonated on shaking in acid solution. However, the introduction of further base functions increases the molecular mass of the base, in turn at the cost of the amounts of waste and the atom economy already mentioned.

The yields, which at <90% are often unsuitable for an industrial process, could also be improved.

It is therefore an object to develop a process for preparing amides or esters from carboxylic acids and an amine or alcohol component in the presence of a 1,3,5-triazine and also of a tertiary amine, optionally in the presence of an organic solvent, in which the added tertiary amine only has a very small molar mass per mole of 1,3,5-triazine used. The total mass of the tertiary amine used shall in particular be considerably below the total mass of the N-methylmorpholine used almost exclusively hitherto and the base shall contain two base functionalities. Furthermore, the new coupling system shall allow high yields to be obtained at a relatively short reaction time.

This object is achieved by a process in which the tertiary amine used is a (bi)cyclic diamine of the general formula I

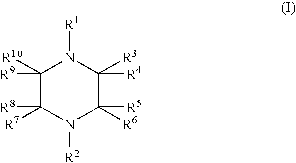

or an adduct of the general formula II formed from it with the triazine component

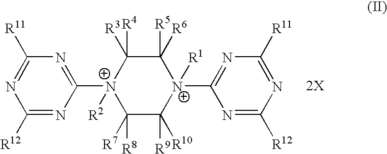

where R¹ and R² are each CH₃ or are together a —(CH₂)₂— bridge, and R³ to R¹² are each independently H, C₁₋₁₀-alkyl, C₁₋₁₀-alkoxy, in particular methoxy, ethoxy, propoxy, butoxy, phenoxy or aryl, in particular C₅₋₃₀-aryl, optionally substituted by one or more C₁₋₁₀-alkyl groups, and 2X is one or more anions to balance the charge, preferably halide ions, for example Cl⁻, Br⁻, I⁻ or HSO₄⁻, or sulfate or organic carboxylate anions, for example acetate, propionate or benzoate, or are any desired mixture of the compounds I and/or II.

In this process, it was found that, surprisingly, the use of the (bi)cyclic diamine having two tertiary amino groups which is essential to the invention together with a 1,3,5-triazine functions as an excellent coupling system and leads to the amides or esters in very good to quantitative yields of generally >80%. The desired products are obtained at a high rate of formation which noticeably exceeds the formation rates known from the prior art. Surprisingly, the (bi)cyclic diamine component essential to the invention may also be used in less than stoichiometric quantities. Even when only 0.5 equivalents of (bi)cyclic diamine are used, the reaction proceeds very effectively.

It is also extremely surprising that the reaction proceeds smoothly with high yields, even though a plurality of possible diamine-triazine adducts having differing charges and chemical properties are conceivable as intermediates owing to the difunctionality of the (bi)cyclic diamine.

The choice of the carboxylic acids is not limited to simple carboxylic acids, but rather encompasses all kinds of carboxylic acids. For instance, the reaction is very efficient when amino acids are preferably used, for example α- and β-amino acids, and preferably enantiomerically pure amino acids, N-protected amino acids, N-protected peptides having at least one free carboxyl group, or else carboxylic acids of the general formula R—COOH where R=C₆₋₁₄-aryl optionally substituted by one or more C₁₋₁₀-alkyl groups, C₁₋₁₇-alkyl and C₃₋₁₄-cycloalkyl. An example of R is (t-butyl)phenyl.

The amine component used may likewise be any kind of amine. In particular, the process is suitable when the amine component used is an amino acid, for example an α- or β-amino acid, preferably in enantiomerically pure form, a C-protected amino acid or C-protected peptide, each having at least one free amino group, or a compound of the general formula R—NH₂ where R=C₆₋₁₄-aryl optionally substituted by one or more C₁₋₁₀-alkyl groups, C₁₋₁₇-alkyl or C₃₋₁₄-cycloalkyl.

The alcohol component used may be any compound having a free hydroxyl group.

The process is thus suitable in particular for preparing peptides by forming the peptide bond in a condensation reaction starting from correspondingly suitable carboxylic acid and amine components. These include N-terminal peptides having an amino function and a protected carboxyl function or C-terminal peptides having a free carboxyl function and a protected amino function. This reaction proceeds particularly efficiently with regard to the formation rate and formation speed. Racemization which presents a considerable problem with existing coupling reagents, for example dicyclohexylcarbodiimide (DCC), does not occur.

The 1,3,5-triazine component is preferably a chlorine-substituted 1,3,5-triazine and has the following general structure:

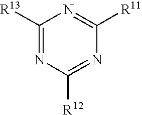

where the radicals R¹¹ and R¹² are each independently O-alkyl having up to 14 carbon atoms, preferably OCH₃, OC₂H₅, O-aryl having up to 14 carbon atoms, alkyl having up to 14 carbon atoms, N(alkyl)₂ having up to 18 carbon atoms, Cl or Br, and R¹³ is Cl.

A particularly suitable 1,3,5-triazine component contemplated by the present invention is 2-chloro-4,6-dimethoxy-1,3,5-triazine (CDMT). However, the reaction also succeeds when other derivatives having a 1,3,5-triazine fragment are used, for example 2,4-dichloro-6-methoxy-1,3,5-triazine or cyanuric chloride.

The cyclic diamine having two tertiary amino groups used is preferably N,N′-dimethyl-1,4-piperazine, although other representatives of this compound class such as bicyclic diazabicyclo[2.2.2]octane (DABCO) or 1,4-diethylpiperazine have also proven extremely useful for the process according to the invention.

The coupling reaction is customarily carried out by reacting a carboxylic acid with an amine or alcohol in the presence of the particular triazine and the (bi)cyclic diamine. Preference is given to initially charging carboxylic acid, then adding the (bi)cyclic diamine having the two tertiary amino groups, followed by the particular triazine component used. Finally, the amine or alcohol component is added. However, the order of addition should not be restricted to this sequence. Rather, it is possible to carry out the reaction with any desired order of addition of the individual components.

In the present process, the reaction is preferably carried out at reaction temperatures of from −80° C. to +150° C., more preferably from −20° C. to +40° C. and in particular from −5° C. to 25° C.

The present invention also contemplates carrying out the reaction in the presence of an organic solvent such as tetrahydrofuran, methyl tert-butyl ether, ethyl acetate, halogenated solvents, for example dichloromethane, or any desired mixtures thereof.

Typically, the reaction works best when the ratio of carboxylic acid to triazine component, depending on the chlorine content of the triazine component, is from 0.50 to 1.50 and preferably from 0.95 to 1.0. The reaction partners carboxylic acid and amine or alcohol component may be used substantially stoichiometrically in a wide range of from 0.2 to 5.0, although preference is given to a ratio of from 0.80 to 1.20; one of these two reaction partners may also be used in excess. The ratio of (bi)cyclic diamine to the triazine component should be from 0.30 to 1.10, in particular from 0.30 to 0.75 and more preferably from 0.47 to 0.53.

As mentioned, instead of adding the 1,3,5-triazine and (bi)cyclic diamine, the adduct formed from these two components and optionally isolated can be added, which likewise forms part of the subject matter of the present invention (cf. formulae II and IV).

According to the invention, adducts having the following special formulae III and V in particular have proven useful:

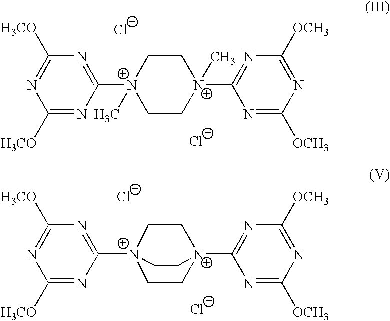

In addition to the preparation process, the present invention also claims the compounds of the formulae (II) to (V).

The novel coupling system using preferably only half-stoichiometric proportions of a (bi)cyclic tertiary diamine and also stoichiometric proportions of a 1,3,5-triazine allows the preparation of amides or peptides in high yields of up to 100%. These yields not only exceed the results from the prior art but furthermore guarantee a substantially reduced amount of waste. For instance, assuming the same yields and using N-methylmorpholine according to the prior art, twice as much waste is produced as in the use according to the invention of N,N′-dimethyl-1,4-piperazine. Furthermore, the absolute amount of waste reduces in comparison to the prior art, since the yields which are obtained by the present process are also higher.

In summary, the present process therefore has the following advantages:

(a) Higher yields compared to the prior art.

(b) Short reaction times, since generally the reactions are already over after 1 hour.

(c) Distinctly smaller amounts of waste of tertiary amine base compared to the prior art (generally <50 -60%).

(d) Improved removal of the product by the possibility of forming a bis-hydrochloride, combined with an improved water solubility.

The present invention claims a process for preparing amides or esters from carboxylic acids and an amine or alcohol component in the presence of a 1,3,5-triazine, optionally in the presence of an organic solvent and of a tertiary amine, by using as tertiary amine a (bi)cyclic diamine or an adduct formed from it with the triazine component in the preferred stoichiometric ratio to the triazine component of from 0.30 to 1.10; the stoichiometric ratio of carboxylic acid to the amine or alcohol component should be from 0.2 to 5.0 and the molar ratio of carboxylic acid to the triazine component from 0.5 to 1.5. Useful carboxylic acid components include amino acids, for example N-protected amino acids and peptides, and useful amine components include (C-protected) amino acids or C-protected peptides. The 1,3,5-triazine used is preferably 2-chloro-4,6-dimethoxy-1,3,5-triazine (CDMT) and the cyclic diamine is N,N′-dimethyl-1,4-piperazine. In addition to this process which may be carried out at temperatures of from −80 to +150° C., and also in the presence of an organic solvent, the present invention also claims adducts of (bi)cyclic diamine and 1,3,5-triazine. In comparison to the prior art, the present process allows higher yields to be obtained in shorter reaction times, and distinctly smaller amounts of waste of tertiary amine base occur.

The examples which follow illustrate these advantages of the process according to the invention:

### EXAMPLE

### Example 1 (Comparative Example)

10 ml of THF were initially charged in a 100 ml three-neck flask equipped with thermometer and 3.00 mmol of 4-tert-butylbenzoic acid were added. 3.05 mmol of N-methylmorpholine were then added dropwise to this mixture with stirring and then 3.03 mmol of 2-chloro-4,6-dimethoxy-1,3,5-triazine (CDMT) were added. This mixture was then stirred for 1 h and 3.0 mmol of benzylamine were added dropwise to this reaction mixture. After stirring for 16 hours, 10 ml of dichloromethane and also 10 ml of an aqueous 5% citric acid solution were added, the phases were then separated and the organic phase was washed in succession with 10 ml of saturated sodium hydrogencarbonate solution and 10 ml of water, dried over sodium sulfate and, after filtration, freed of solvent on a rotary evaporator. The N-benzyl-4-tert-butylbenzamide was obtained as a white solid in a yield of 67%.

### Example 2

10 ml of THF were initially charged in a 100 ml three-neck flask equipped with thermometer and 3.00 mmol of 4-tert-butylbenzoic acid were then added. 1.55 mmol of 1,4-dimethylpiperazine were then added dropwise to this mixture with stirring and then 3.03 mmol of 2-chloro-4,6-dimethoxy-1,3,5-triazine were added. This mixture was stirred for 1 h and 3.0 mmol of benzylamine were added dropwise to this reaction mixture. After stirring for 16 hours, 10 ml of dichloromethane and also 10 ml of an aqueous 5% citric acid solution were added, the phases were then separated and the organic phase was washed in succession with 10 ml of saturated sodium hydrogencarbonate solution and 10 ml of water, dried over sodium sulfate and, after filtration, freed of solvent on a rotary evaporator. The N-benzyl-4-tert-butylbenzamide was obtained as a white solid in a yield of 88%.

### Example 3

130 ml of THF were initially charged in a 500 ml three-neck flask equipped with thermometer and 30.0 mmol of 4-tert-butylbenzoic acid were then added. 15.5 mmol of 1,4-dimethylpiperazine were then added dropwise to this mixture with stirring and then 30.3 mmol of 2-chloro-4,6-dimethoxy-1,3,5-triazine were added. This mixture was then stirred for 1 h and 30.0 mmol of benzylamine dissolved in 5 ml of THF were added dropwise to this reaction mixture. After stirring for 16 hours, 130 ml of dichloromethane and also 100 ml of an aqueous 5% citric acid solution were added and the phases were then separated. The aqueous phase was again extracted using 100 ml of dichloromethane, the collected organic phases were washed in succession with 80 ml of saturated sodium hydrogencarbonate solution and 45 ml of water, then dried over sodium sulfate and, after filtration, freed of solvent on a rotary evaporator. The N-benzyl-4-tert-butylbenzamide was obtained as a white solid in a yield of >99%.

### Example 4

10 ml of THF were initially charged in a 100 ml three-neck flask equipped with thermometer and 3.00 mmol of 4-tert-butylbenzoic acid were then added. 1.55 mmol of 1,4-diazabicyclo[2.2.2]octane were then added dropwise to this mixture with stirring and then 3.03 mmol of 2-chloro-4,6-dimethoxy-1,3,5-triazine were added. This mixture was then stirred for 1 h and 3.0 mmol of benzylamine were added dropwise to this reaction mixture. After stirring for 16 hours, 10 ml of dichloromethane and also 10 ml of an aqueous 5% citric acid solution were added and the phases were then separated. The organic phase was washed in succession with 10 ml of saturated sodium hydrogencarbonate solution and 10 ml of water, then dried over sodium sulfate and, after filtration, freed of solvent on a rotary evaporator. The N-benzyl-4-tert-butylbenzamide was obtained as a white solid in a yield of 66%.

### Example 5

30 ml of THF were initially charged in a 100 ml three-neck flask equipped with thermometer and 6.00 mmol of pivalic acid were then added. 3.2 mmol of 1,4-dimethylpiperazine were then added dropwise to this mixture with stirring and then 6.5 mmol of 2-chloro-4,6-dimethoxy-1,3,5-triazine were added. This mixture was then stirred for 2 h and 6.5 mmol of 2-phenylethylamine were slowly added dropwise to this reaction mixture. After stirring for 3 hours, 30 ml of dichloromethane and also 50 ml of an aqueous 5% citric acid solution were added, the phases were then separated and the aqueous phase was washed again with 2×20 ml dichloromethane. The collected organic phases were washed in succession with 40 ml of water, 50 ml of saturated sodium hydrogencarbonate solution and again with 40 ml of water, then dried over sodium sulfate and, after filtration, freed of solvent on a rotary evaporator. The N-phenylethylpivalamide was obtained as a white solid in a yield of 90%.

### Example 6

7.65 mmol of 1,4-dimethylpiperazine were added dropwise with cooling to a stirred solution of 2.66 g of 2-chloro-4,6-dimethoxy-1,3,5-triazine (CDMT) and 3.32 g of Boc-Ser-OH (BOC-serine: M=205.21) in 15 ml of dichloromethane in such a manner that the internal temperature was from −5 to 0° C. The stirring was then continued at 0° C. until all the CDMT had reacted (about 1 hour). A mixture consisting of 5.69 g of H-Val-OBzl*p-tosylate (benzyl valinate-p-toluenesulfonate: M=379.48) and 0.89 g of 1,4-dimethylpiperazine in 7.5 ml of dichloromethane was then added dropwise to this reaction mixture at from −5 to 0° C., before it was stirred at 0° C. for a further 2 hours. The stirring was then continued at room temperature for a further 14 hours, the solvent was then removed on a rotary evaporator and the residue was taken up in 45 ml of ethyl acetate. The resulting suspension was then washed in succession with 15 ml of water, 15 ml of 10% citric acid, 15 ml of water, 15 ml of saturated sodium hydrogencarbonate solution and finally 15 ml of water. The organic phase was finally dried over magnesium sulfate, then filtered, concentrated under reduced pressure and recrystallized from ethyl acetate/petroleum ether. The product was obtained in 85% yield.

### Example 7

A 100 ml three-neck flask was charged with 6 mmol of 4-tert-butylbenzoic acid and 6.06 mmol of CDMT in 20 ml of THF, and 3.1 mmol of dimethylpiperazine were added dropwise to this mixture with stirring. After 1 hour, 20 ml of methanol were added and the mixture was stirred for 16 hours. The solvent was then distilled off, 20 ml of methylene chloride were added to the residue obtained and extraction was effected using 10 ml of 5% citric acid. The organic phase was washed first with 30 ml of saturated sodium hydrogencarbonate solution and then with 30 ml of water, then dried over sodium sulfate and, after filtration, the solvent was distilled off. In this way, the desired ester was obtained in a yield of 85%.

### Example 8

10 ml of THF were initially charged in a 100 ml three-neck flask equipped with thermometer and 3.00 mmol of tert-butylbenzoic acid were then added. 3.05 mmol of 1,4-dimethylpiperazine were then added dropwise to this mixture with stirring and then 3.03 mmol of 2-chloro-4,6-dimethoxy-1,3,5-triazine were added. This mixture was stirred for 1 h before 3.0 mmol of benzylamine were added dropwise to this reaction mixture. After stirring for 16 hours, 10 ml of dichloromethane and also 10 ml of an aqueous 5% citric acid solution were then added and the phases were then separated. The organic phase was washed in succession with 10 ml of saturated sodium hydrogen carbonate solution and 10 ml of water, then dried over sodium sulfate and, after filtration, freed of solvent on a rotary evaporator. The N-benzyl-tert-butylbenzamide was obtained as a white solid in a yield of 93%.

Discussion:

The superiority of the present process, especially with regard to the yield, in comparison to the existing synthesis methods is exhibited, for instance, in the direct comparison with the existing methods using N-methylmorpholine (see comparative example 1) using the example of the coupling reaction of tert-butylbenzoic acid and benzylamine as the carboxylic acid and amine component respectively. For instance, the existing system “CDMT (1.01 equiv.)/N-methylmorpholine (1.017 equiv.)” provides a yield of only 67% (see comparative example 1), whereas the coupling system according to the invention, for example consisting of CDMT (1.01 equiv.) and distinctly reduced amounts of 1,4-dimethylpiperazine (0.517 equiv.) allows a greatly increased yield of 88% to be achieved (example 2) which, when the addition technique is changed and the workup is optimized in an increased batch, can even be increased to >99% (example 3).

In addition to the ecological advantages, such as smaller amounts of waste of base, and also the optimized atom economy, the present coupling system consisting of a 1,3,5-triazine and a cyclic diamine thus provides a coupling system having improved chemical efficiency. In addition, the reaction time could be considerably reduced: for instance, even after (less than) 1 hour of reaction time, quantitative conversion is observed. When 1.017 equivalents of the cyclic diamine, 1,4-dimethylpiperazine, are used instead of 1.017 equivalents of N-methylmorpholine of the prior art (see comparative example 1), an increased yield of 93% (example 8) is obtained instead of the 67% yield as in the prior art (comparative example 1).

The coupling reaction also proceeds very efficiently with (bi)cyclic diamines having two tertiary amino groups other than 1,4-dimethylpiperazine. For instance, when diazabicyclo[2.2.2]octane (DABCO) is used, the desired coupling product is obtained in 66% yield (example 4). Example 5 documents that the novel coupling reagent can also be efficiently used for the coupling of aliphatic carboxylic acids (yield: 90%). In addition, the suggested process is also excellently suitable for coupling unprotected or N-protected amino acids or corresponding peptides. Interestingly, even the presence of additional functional groups is tolerated, as example 6 shows. For instance, the coupling with the novel system proceeds highly efficiently with 85% yield in the synthesis of the coupling product starting from BOC-Ser-OH and H-Val-OBzl*tosylate (example 6).

What is claimed is:

1. A process for preparing amides or esters from carboxylic acids and an amine or alcohol component in the presence of a 1,3,5-triazine and also of a tertiary amine or a triazine-amine adduct, optionally in the presence of an organic solvent, characterized in that as the tertiary amine a (bi)cyclic diamine of the general formula I 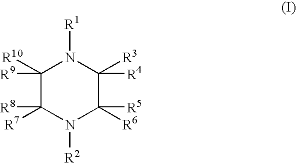
or an adduct of the general formula II formed from it with the triazine component 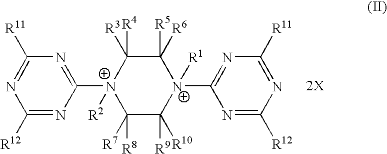
where R¹ and R² are each CH₃ or are together a —(CH₂)₂— bridge, and R³ to R¹² are each independently H, C₁₋₁₀-alkyl, C₁₋₁₀-alkoxy, in particular methoxy, ethoxy, propoxy, butoxy, phenoxy or aryl, and 2X is one or more anions, preferably halide ions, for example Cl⁻, Br⁻, I⁻ or HSO₄⁻, or sulfate or organic carboxylate anions, or any desired mixture of the compounds I and/or II is used.

2. The process as claimed in claim 1, characterized in that the carboxylic acid component used is an amino acid, preferably an enantiomerically pure amino acid, or a derivative thereof such as an N-protected amino acid, N-protected peptide having at least one free carboxyl group, or else a carboxylic acid of the general formula R—COOH where R=C₆₋₁₄-aryl optionally substituted by one or more C₁₋₁₀-alkyl groups, C₁₋₁₇-alkyl or C₃₋₁₄-cycloalkyl.

3. The process as claimed in one of claims 1 and 2, characterized in that the amine component used is an amino acid, preferably an enantiomerically pure amino acid, or a derivative thereof such as a C-protected amino acid or C-protected peptide, each having at least one free amino group, or a compound of the general formula R—NH₂ where R=C₆₋₁₄-aryl optionally substituted by one or more C₁₋₁₀-alkyl groups, C₁₋₁₇-alkyl or C₃₋₁₄-cycloalkyl.

4. The process as claimed in one of claims 1 to 3, characterized in that a chlorine-substituted 1,3,5-triazine component is used.

5. The process as claimed in one of claims 1 to 4, characterized in that the 1,3,5-triazine used is 2-chloro-4,6-dimethoxy-1,3,5-triazine (CDMT).

6. The process as claimed in one of claims 1 to 5, characterized in that the cyclic diamine used is N,N′-dimethyl-1,4-piperazine.

7. The process as claimed in one of claims 1 to 5, characterized in that the bicyclic diamine used is diazabicyclo[2.2.2]octane (DABCO).

8. The process as claimed in one of claims 1 to 7, characterized in that the carboxylic acid component is initially charged and then the cyclic diamine, the triazine component and finally the amine or alcohol component are added.

9. The process as claimed in one of claims 1 to 8, characterized in that the reaction is carried out at temperatures of from −80 to +150° C., preferably from −20 to +40° C. and more preferably from −5 to +25° C.

10. The process as claimed in one of claims 1 to 9, characterized in that the reaction is carried out in the presence of an organic solvent such as tetrahydrofuran, methyl tert-butyl ether, ethyl acetate, halogenated solvents, for example dichloromethane, or any desired mixtures thereof.

11. The process as claimed in one of claims 1 to 10, characterized in that the stoichiometric ratio of the cyclic diamine to the triazine component is from 0.30 to 1.10, in particular from 0.30 to 0.75 and more preferably from 0.47 to 0.53.

12. The process as claimed in one of claims 1 to 11, characterized in that the ratio of carboxylic acid to amine or alcohol component is from 0.2 to 5.0 and preferably from 0.80 to 1.20.

13. The process as claimed in one of claims 1 to 12, characterized in that the molar ratio of carboxylic acid to triazine component is from 0.5 to 1.5 and preferably from 0.95 to 1.0.

14. The process as claimed in one of claims 1 to 13, characterized in that an adduct of the formula 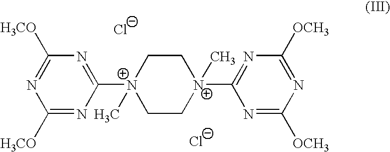
is used.

15. The process as claimed in one of claims 1 to 13, characterized in that an adduct of the formula (V)

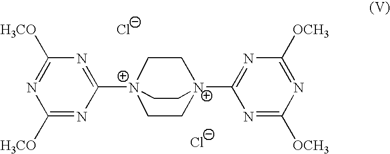

16. A compound of the general formula (IV) 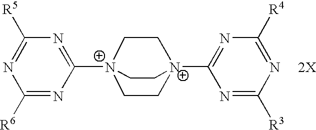
where R¹ to R⁴ are each independently H, C₁₋₁₀-alkyl, C₁₋₁₀-alkoxy, in particular methoxy, ethoxy, propoxy, butoxy, phenoxy or aryl, and 2X is one or more anions, preferably halide ions, for example Cl⁻, Br⁻, I⁻ or HSO₄⁻, or sulfate or organic carboxyl are anions.

17. The compound as claimed in claim 16 of the formula (V) [chemical structure]
